(19) United States
(12) Plant Patent Application Publication
(10) Pub. No.: US 2017/0303458 P1
(43) Pub. Date: Oct. 19, 2017

(54) Ornithogalum plant named 'BCOR-09.002'

(50) ;

(71) Applicant: Rodolfo Valdoz Bautista, Half Moon Bay, CA (US)

(72) Inventor: Rodolfo Valdoz Bautista, Half Moon Bay, CA (US)

(21) Appl. No.: 14/999,282

(22) Filed: Apr. 18, 2016

(51) Int. Cl. A01H 5/02

(52) U.S. Cl. CPC A01H 5/02
USPC PLT/462

(57) ABSTRACT

A new and distinct cultivar of Omithogalum dubium named ‘BCOR-09.002’ resulted from a controlled hybridization of commercial varieties. The cultivar ‘BCOR-09.002’ has R.H.S. 11 B (yellow group) tepals, erect and strong racemes which resist bending under low light conditions, a long blooming habit and broad, channeled leaves.

Botanical classification: Ornithogalum dubium ‘BCOR-09.002’.

Variety denomination: ‘BCOR-09.002’.

### BACKGROUND OF THE INVENTION

This invention relates to a new and distinct cultivar of the Lilaceae family. The botanical name of the plant is Ornithogalum dubium ‘BCOR-09.002’.

The new cultivar is the result of controlled crossing of commercial varieties.

The new variety ‘BCOR-09.002’ is differentiated from other commercial varieties known to the inventor as shown in the table below.

TABLE 1

| | ‘Lourdes’ | | ‘Chesapeake | | |
|---|---|---|---|---|---|
| | Commercial | ‘Bethlehem’ | Snowflake’ | ‘Orangezicht’ | New Variety |
| | Variety | USPP 21,427 | USPP 13,200 | USPP 12,226 | BCOR-09.002 |
| Rachis/Raceme | 40 to 50 cm | 43 cm | 20-30 cm | 26 cm | 25 to 40 cm |
| height | | | | | |
| Rachis color | Varies between | R.H.S. 146A | Not reported | Not reported | Mature stem |
| | R.H.S. 146B, | | | | R.H.S. 146A |
| | R.H.S. 147B, | | | | (green group) |
| | or | | | | Immature stem |
| | R.H.S. 145D | | | | R.H.S. 146D |
| | | | | | (yellow-green |
| | | | | | group) |
| Reflexed leaves | No | Yes | Not reported | Leaves lay flat | Most upright; |
| | | | | on ground | some slightly |
| | | | | | reflexed |
| Leaf color | R.H.S. 137A | R.H.S. 146A | R.H.S. 143B | Upper surface | Immature |
| | | | | R.H.S. 137A | 137C (green |
| | | | | or 147A | group); Mature |
| | | | | | 144A (yellow- |
| | | | | | green group) |
| Floret diameter | 30 mm | 70 mm | 50-60 mm | 50-60 mm | 40 mm |
| Tepal height | 30 mm | 36 mm | 20-25 mm | 25-27 mm | 30 mm |
| Tepal Color | R.H.S. 25A | R.H.S. 155C | R.H.S. 155A-D | Varies | R.H.S. 11B |
| | | | | between | |
| | | | | R.H.S. 25A, | |
| | | | | R.H.S. 25D, or | |
| | | | | R.H.S. 28A | |
| Tepal Spot | R.H.S. 152B | R.H.S. 152A | R.H.S. 147A | R.H.S. 200B | R.H.S. 152A |
| Color | | | | | |

The new variety is suitable for commercial sale as a potted plant.

The new cultivar ‘BCOR-09.002’ has been asexually reproduced under controlled environmental conditions at a nursery in Half Moon Bay, Calif. under the direction of the inventor with its distinguishing characteristics remaining stable.

Asexual reproduction was first accomplished when vegetative cuttings were taken from the initially selected plant. Examination of asexually reproduced, successive generations grown in a nursery at Half Moon Bay, Calif. shows that the combination of characteristics as herein disclosed for ‘BCOR-09.002’ remains firmly fixed.

### DESCRIPTION OF THE DRAWINGS

The accompanying drawings consist of color photographs that show the typical plant form, including the inflorescence, foliage, and sepals.

FIG. 1 is a view of the new variety showing its form.

FIG. 2 is a top view of a multiple inflorescences of the new variety.

FIG. 3 is a close-up view of the base of the plant of the new variety.

FIG. 4 is a close-up view of the top of the inflorescence of the new variety.

FIG. 5 is a close-up view of a number of inflorescences of the new variety.

FIG. 6 is a close-up view of several new florets of the new variety.

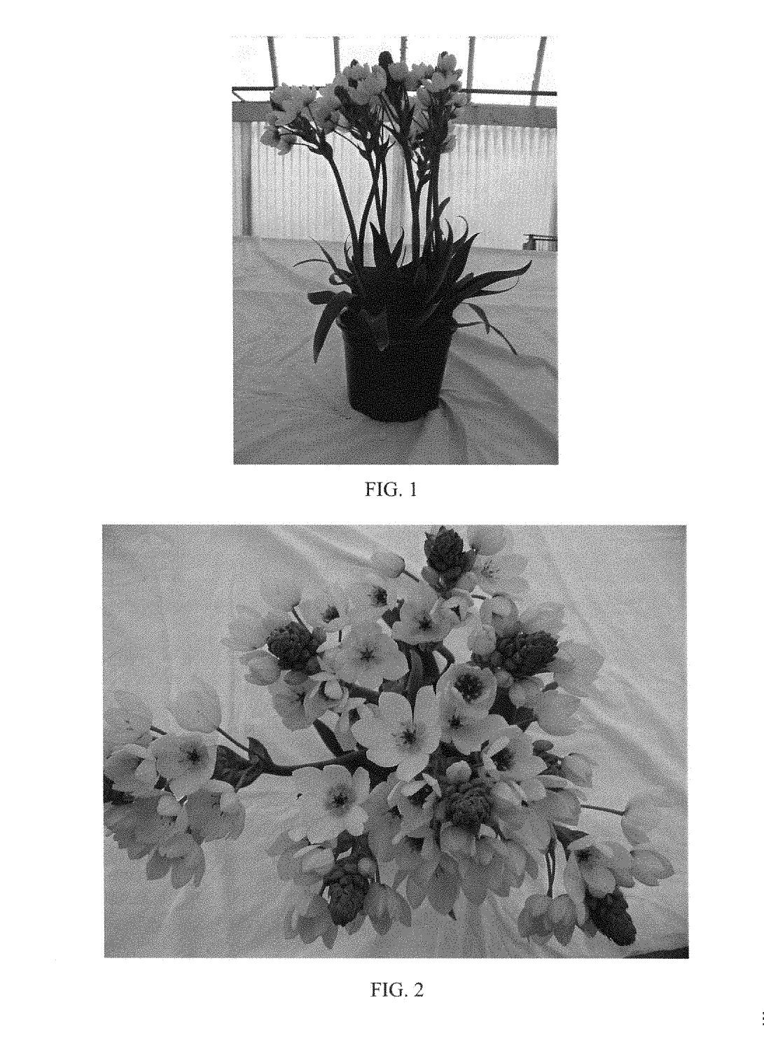

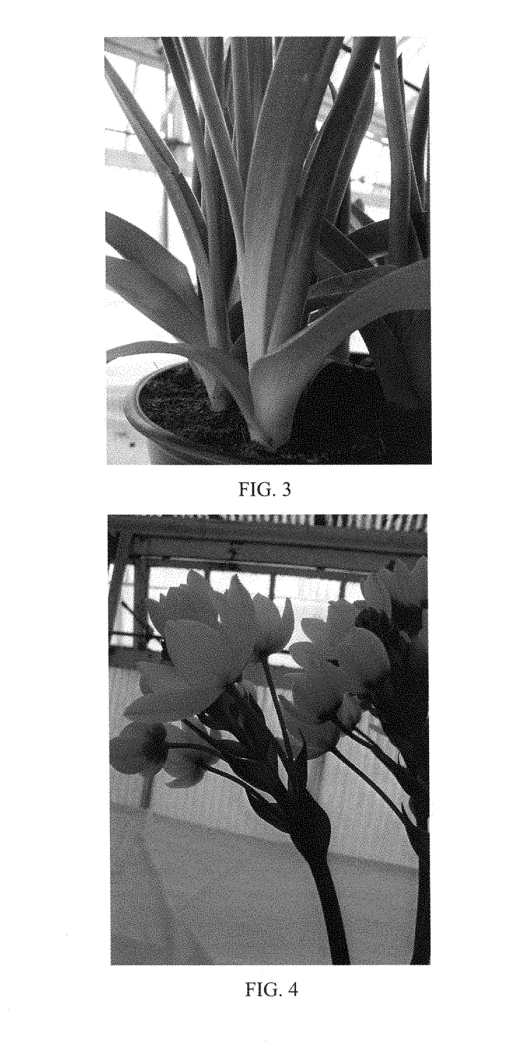

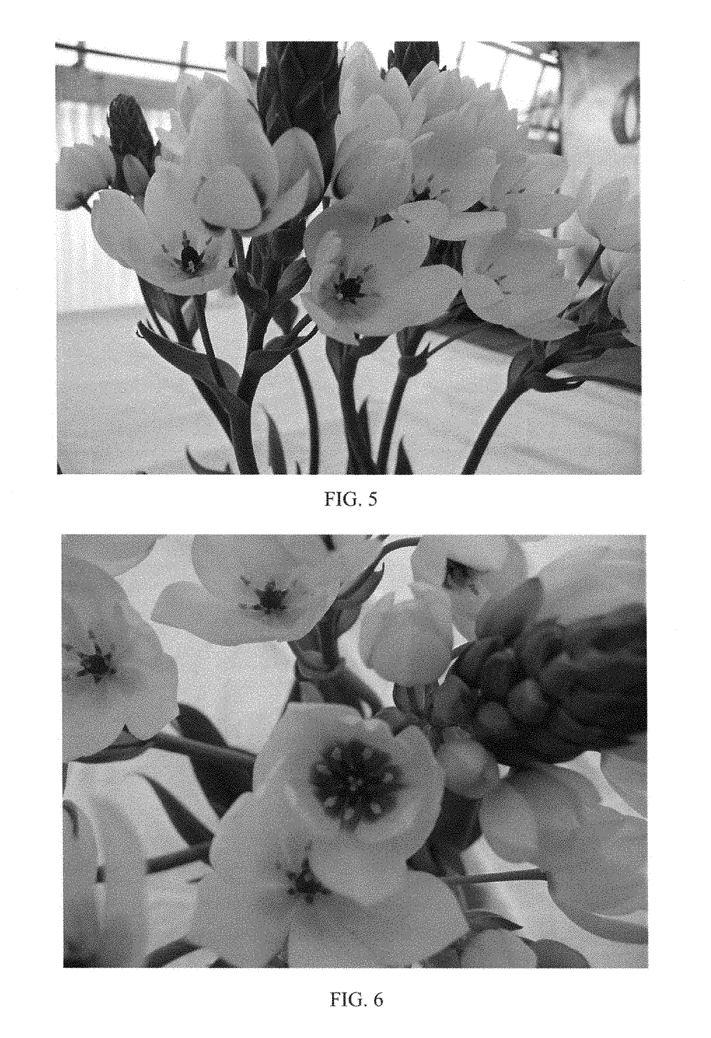

### DESCRIPTION OF THE NEW PLANT

The plants shown in the figures were grown in 6″ pots. The plants were started in a greenhouse from leaf cuttings.

‘BCOR-09.002’ has not been observed under all possible environmental conditions. The phenotype may vary significantly with variations in environment such as temperature, light intensity and day length. Color determinations were made with The Royal Horticultural Society (R.H.S.) Colour Chart.
The plant:
   Origin.—Controlled cross of commercial varieties.
   Form.—Spreading basal leaves with upright inflorescences on tall rachis. A typical plant with a mature inflorescence that is ready for sale at ten months is approximately 25 to 40 cm high when grown in a 6″ pot with appropriate soil amendments. Plants shown are 40 cm high.
   Growth.—Upright rachis with somewhat reflexed leaves. Inflorescence is large and showy.
   Rachis.—The rachis is upright and glaucous. Its color varies between R.H.S. 146 D (yellow-green group) when younger and R.H.S. 146 A (yellow-green group), at maturity of the inflorescence. Diameter is 9 mm. The length of the rachis to the first floret and bract pair is 25 cm. Rachis or stem at its base is 10 mm in diameter.
   Foliage.—Usually three to five leaves surround each rachis on a mature plant ready for commercial sale. Leaves are whorled at base of stem. Shape of leaf. — Fleshy, channel-shaped, lanceolate. Margins are entire. Apex is acute. Texture. — Glaucescent. Color. — Mature leaves are R.H.S. 144 A (yellow group) on both abaxial and adaxial surfaces. Large leaves are 17 cm long and 4.5 cm wide. Immature leaves are R.H.S. 137 C (green group).
Inflorescence:
   Form.—Corymbose racemes. Indeterminate. Approximately 50 or more individual florets per inflorescence on pedicels that elongate as the florets opens, and on average are as long as 120 mm for mature florets. Apical buds on developed inflorescences are predominately green — R.H.S. 145 D (yellow-green group), with their apex being R.H.S. 146 B (yellow-green group), 0.9-1.0 cm tall and have diameter of 3 mm wide. The rachis for the inflorescence is strong and upright. The pedicels are glabrous and similar in color to the rachis at their point of attachment.
   Size of inflorescence.—Large. A typical inflorescence at this age, as shown in the figures can be 18 cm high.
   Shape.—Basal florets opening first with pedicels that elongate that give the raceme a corymbose appearance as the inflorescence ages.
   Appearance.—Showy.
Florets:
   General.—The florets at the base of the inflorescence open first, and open more and more as the inflorescence ages. Florets are perfect and complete. Perianth is not keeled. Each floret is associated with a bract that can be as wide as 20 mm and as wide as 15 mm and as high as 27 mm when floret is beginning to open, and has tips that are R.H.S. 137 B (green group). Tips of bract are darkest part of bract. Bracts retain color. The bracts are ovate with narrowly acute apexes and relatively wide bases and reflexed/curled edges around rachis. Perianth: There are 6 ovate tepals that are spotted at their bases on both their abaxial and adaxial surfaces. The spot is R.H.S. 152 A (yellow-green group) on the adaxial surface. The abaxial spot is R.H.S. 143 A (green group). Spot fades as floret ages. The tepals are predominantly R.H.S. 11 B (yellow group). Floret diameter: 40 mm. Tepals of mature florets are typically 30 mm high, 20 mm wide at their widest point, and 5 mm wide at their attachment. Immature tepals have spotted base, the spot is approximately 7 mm high and extends the width of the tepal at the base. Apex shape of the tepal is abruptly acuminate.
   Stamens.—6 stamens (two sets of 3). Pollen is R.H.S. 14 A (yellow-orange group). Plant produces abundant pollen. Filament is wide with basal wings. Filament is 3 mm wide at base and 4 mm high. Anther is 3 mm high and basally attached. Filament base is R.H.S. 155 A (white group). Anther is R.H.S. 152 B (yellow-green group) before opening to reveal pollen.
   Stigma.—Generally, a three-pronged stigma on one pistil per floret. The style is 2 mm long. The stigma is approximately 2 mm long.
   Ovary.—The ovary is three-celled with axile placentae. The top of the ovary is R.H.S. 139 A (green group). The ovary is 7 mm high and has a diameter of 4 mm.
   Fragrance.—Florets product a very mild citrus fragrance.

I claim:

1. A new and distinct Ornithogalum plant named ‘BCOR-09.002’ substantially as herein shown and described.
